(19) United States
(12) Patent Application Publication
(10) Pub. No.: US 2025/0269017 A1
(43) Pub. Date: Aug. 28, 2025

(54) SYSTEMS, DEVICES AND METHODS FOR APPLYING ANTI-VEGF COMPOUNDS AND USING SUCH COMPOUNDS TO TREAT SKIN CONDITIONS

(71) Applicant: Cellviva, Inc., San Jose, CA (US)

(72) Inventors: Eric F. BERNSTEIN, Gladwyne, PA (US); Randal PHAM, Los Gatos, CA (US); Dale KOOP, Woodside, CA (US)

(21) Appl. No.: 19/206,844

(22) Filed: May 13, 2025

Related U.S. Application Data
(63) Continuation of application No. 16/549,426, filed on Aug. 23, 2019, now abandoned.
Application No. 16/549,426 is a continuation-in-part of application No. 15/700,992, filed on Sep. 11, 2017, now Pat. No. 10,556,008.
Application No. 15/700,992 is a continuation of application No. 14/861,943, filed on Sep. 22, 2015, now Pat. No. 9,757,452.
Application No. 14/861,943 is a division of application No. 14/261,198, filed on Apr. 24, 2014, now Pat. No. 9,161,978.
Application No. 14/261,198 is a division of application No. 13/830,819, filed on Mar. 14, 2013, now Pat. No. 8,747,852.
(60) Provisional application No. 61/746,778, filed on Dec. 28, 2012.
(60) Provisional application No. 62/721,656, filed on Aug. 23, 2018.

(51) Int. Cl. A61K 39/395; A61K 31/196; A61K 31/573; A61K 31/58; A61K 39/00; A61K 45/06; C07K 16/22

(52) U.S. Cl. CPC A61K 39/3955; A61K 31/196; A61K 31/573; A61K 31/58; A61K 45/06; C07K 16/22; A61K 2039/505

(57) ABSTRACT

A method for treating an inflammatory or vascular condition by transdermally regulating inflammation in a target area of the body having an inflammatory or vascular condition by topically applying to the skin of the target area a therapeutically effective amount of at least one anti-VEGF containing compound. A device, which may be comprises a transdermal patch, a bandage, a paint, an atomized spray, for treating an inflammatory or vascular condition is also disclosed. The device is configured to transdermally apply to the skin of the target area a therapeutically effective amount of at least one anti-VEGF containing compound that regulates inflammation in the target area.

### CROSS-REFERENCE TO RELATED APPLICATIONS

This is a continuation of U.S. application Ser. No. 16/549,426 filed Aug. 23, 2019, which is a continuation-in-part of U.S. application Ser. No. 15/700,992 filed Sep. 11, 2017, now U.S. Pat. No. 10,556,008, which is a continuation of U.S. application Ser. No. 14/861,943 filed Sep. 22, 2015, now U.S. Pat. No. 9,757,452, which is a divisional of U.S. application Ser. No. 14/261,198 filed Apr. 24, 2014, now U.S. Pat. No. 9,161,978 which is a divisional of U.S. application Ser. No. 13/830,819 filed Mar. 14, 2013, now U.S. Pat. No. 8,747,852, which claims priority to U.S. Provisional Application No. 61/746,778, filed Dec. 28, 2012; this application also claims priority to U.S. Provisional Application No. 62/721,656 filed Aug. 23, 2018, each of which is incorporated herein by reference in its entirety.

### TECHNICAL FIELD

The present disclosure relates generally to systems, devices and methods of applying anti-vascular endothelial growth factor (anti-VEGF) to the skin for treating various skin conditions. The present disclosure also relates to delivery systems for applying the disclosed compounds to the skin, including transdermal patches and bandages comprising anti-VEGF and methods of using the same to treat various skin conditions.

### BACKGROUND

Anti-vascular endothelial growth factor therapy, also known as “anti-VEGF” therapy or anti-VEGF medication, is the use of medications that block vascular endothelial growth factor. This is done in the treatment of certain cancers and in age-related macular degeneration. They can involve monoclonal antibodies such as bevacizumab, antibody derivatives such as ranibizumab (Lucentis), or orally-available small molecules that inhibit the tyrosine kinases stimulated by VEGF: lapatinib, sunitinib, sorafenib, axitinib and pazopanib. Some of these therapies target VEGF receptors rather than the VEGFs. Both antibody-based compounds and the first three orally available compounds are commercialized with the latter two (axitinib and pazopanib) in clinical trials.

Bevacizumab is a 149-kD humanized monoclonal antibody that inhibits vascular endothelial growth factor (VEGF-A), a signal protein that stimulates angiogenesis and vasculogenesis for neovascular age-related macular degeneration AMD. While bevacizumab received FDA approval for use in the management of various cancers, compounded bevacizumab has been used off label in the treatment of ophthalmic conditions including AMD since May 2005. Today, bevacizumab is successfully used to also treat diabetic retinopathy, central retinal vein occlusion, neovascular glaucoma, and retinopathy of prematurity, in addition to a host of other less common eye diseases.

The application of AVEGF and other means for slowing or preventing angiogenesis can play a role in many diseases and injuries where excessive angiogenesis can have undesired effects including scar formation, swelling, keloids, prolonged redness and many other conditions listed below.

The disclosed systems, devices are methods for treating inflammatory skin diseases and other skin conditions are directed to overcoming one or more of the problems set forth above and/or other problems of the prior art.

### SUMMARY

In an embodiment, there is disclosed a method for treating an inflammatory or vascular condition by transdermally regulating inflammation in a target area of the body having an inflammatory or vascular condition by topically applying to the skin of the target area a therapeutically effective amount of at least one anti-VEGF containing compound.

In another embodiment, there is disclosed a device, such as a transdermal patch, a bandage, a paint, an atomized spray, for treating an inflammatory or vascular condition is also disclosed. The device is configured to transdermally apply to the skin of the target area a therapeutically effective amount of at least one anti-VEGF containing compound that regulates inflammation in the target area.

Aside from the subject matter discussed above, the present disclosure includes a number of other features such as those explained hereinafter. Both the foregoing description and the following description are exemplary only.

### BRIEF DESCRIPTION OF THE DRAWINGS

The accompanying FIGURES are incorporated in and constitute apart of this specification.

FIGS. 1A-1D shows various designs for a transdermal patch including a matrix (FIG. 1A), a reservoir (FIG. 1B), a multi-laminate (FIG. 1C) and drug-in-adhesive (FIG. 1D) design.

### DETAILED DESCRIPTION

### Definitions

As used herein, the term “subject” means any mammal, and in particular, a human, and can also be referred to, e.g., as an individual or patient.

As used herein, an “anti-VEGF agent” means an inhibitor of VEGF signaling. Anti-VEGF agents include antibodies (e.g., bevacizumab), antibody fragments (e.g., an antibody light chain (VL), an antibody heavy chain (VH), a single chain antibody (scFv), an F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means), fusion proteins, peptide, nucleic acids (e.g., siRNA, shRNA), and other small molecules, etc. that disrupt the interaction between VEGF (VEGF-A) and its receptor (VEGFR-1/VEGFR-2). Other, non-limiting examples of anti-VEGF agents encompassed by the present disclosure are provided herein below.

As used herein, the term “adjacent to”, e.g., in the context of applying or injecting an anti-VEGF adjacent to or near the site of new blood vessel growth, means proximate to (e.g., within about 0.1 mm, 0.2 mm, 0.3 mm, 0.4 mm, 0.5 mm, 1 mm, 2 mm, 3 mm, 4 mm, or 5 mm from the site of blood vessel growth).

As used herein the terms “therapeutically effective” and “effective amount”, used interchangeably, applied to a dose or amount refer to a quantity of a composition, compound or pharmaceutical formulation that is sufficient to result in a desired activity upon administration to a subject in need thereof. Within the context of the present invention, the term “therapeutically effective” refers to that quantity of a composition, compound or pharmaceutical formulation that is sufficient to reduce, eliminate or delay at least one symptom of a disease or condition specified herein. When a combination of active agents is administered, the effective amount of the combination, or individual agents, may or may not include amounts of each agent that would have been effective if administered individually. The dosage of the therapeutic formulation will vary, depending upon the nature of the disease or condition, the patient's medical history, the frequency of administration, the manner of administration, the clearance of the agent from the host, and the like. The initial dose may be larger, followed by smaller maintenance doses. The dose may be administered, e.g., weekly, biweekly, daily, semi-weekly, etc., to maintain an effective dosage level.

Therapeutically effective dosages in the methods described herein can be determined by the treating physician. For example, the physician may begin treatment using manufacturer-recommended doses for the anti-VEGF agent, and make adjustments based on the physician's observations of the effect of treatment. Further guidance is provided herein and in the Examples. In addition, clinical trials can be conducted to determine the doses that are effective to produce statistically significant treatment effects when a population of patients is treated.

As used herein “combination therapy” means the treatment of a subject in need of treatment with a certain composition or drug in which the subject is treated or given one or more other compositions or drugs for the disease in conjunction with the first and/or in conjunction with one or more other therapies, such as, e.g., surgery. Such combination therapy can be sequential therapy wherein the patient is treated first with one treatment modality (e.g., drug or therapy), and then the other (e.g., drug or therapy), and so on, or all drugs and/or therapies can be administered simultaneously. In either case, these drugs and/or therapies are said to be “co-administered.” It is to be understood that “co-administered” does not necessarily mean that the drugs and/or therapies are administered in a combined form (i.e., they may be administered separately or together to the same or different sites at the same or different times).

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “regulating inflammation” is intended to mean suppressing unwanted inflammatory responses, enhancing beneficial inflammatory responses, or otherwise altering the inflammatory response to beneficially treat the described disorders or conditions.

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; and/or (2) inhibiting the state, disorder or condition, including arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease. The benefit to a subject to be treated is either statistically significant or at least perceptible to the patient or to the physician.

As used herein, “target area” is intended to mean the area of the body or skin to be treated because it is the location of, adjacent to or near the site of a vascular or inflammation condition, such as new blood vessel growth associated with one or more of the diseases/conditions described herein.

As used herein, “Lipid-based nanocarriers” are intended to include liposomes and micelles, which are biocompatible.

As used herein, “Liposomes” are intended to mean FDA-approved spherical structures comprising phospholipid bilayers with an enclosed aqueous phase that can carny at least the anti-VEGF compounds described herein.

As used herein, “Micelles” are intended to mean FDA-approved spherical structures comprising lipid monolayers with a hydrophilic shell enclosing a hydrophobic core.

As used herein, “FLT-1” is intended to describe a vascular endothelial growth factor receptor 1 (VEGFR-1), which is a high-affinity tyrosine kinase receptor for VEGF and is expressed almost exclusively on vascular endothelial cells.

### Inflammatory Skin Diseases

In one embodiment, treating includes modulating conditions where increased or altered vasculature is an important component, and thus would benefit from the therapeutic treatment with AVEGF compounds described herein. Thus, methods and devices are provided for treating inflammatory skin diseases, other skin conditions and injuries, abrasions, and surgery that can lead to scars, and prolonged erythema, where increased or remodeled vasculature plays a role in treating inflammatory skin diseases.

There are a variety of modulating conditions where increased or altered vasculature is an important component, and thus would benefit from the therapeutic treatment with AVEGF compounds described herein. These include, but are not limited to: (a) photoaging, (b) skin being treated with dermal or sub-dermal fillers, (c) skin exposed to ionizing radiation, (d) skin exposed to UV-radiation, (e) wounded skin (acute and chronic), (d) vascular conditions like port-wine stains (pre- and post-treatment) which can recur or even proliferate without treatment, as port-wine stains often grow or thicken with time. Other vascular conditions that can be treated include cherry angiomas, hemangiomas, spider angiomas (nevus araneuses), and rosacea, (e) congenital conditions where vessels are key, including but not limited to Hereditary Hemorrhagic Telangiectasia, which is also known as Osler-Randu, (f) acquired conditions where vessels are key-CREST form of Scleroderma, (g) any connective tissue disease such as: rheumatoid arthritis, lupus, scleroderma, Sjogrens's syndrome, Raynaud's syndrome and disease, and others, and (h) acute and chronic wounds.

In one embodiment, there is described using AVEGF with one or more treatment of the skin. For example, in various embodiments there are described methods of treating the skin with the disclosed AVEGF in conjunction with or combination with other skin treatments, such as, but not limited to: in conjunction with post-filler administration; in combination with laser hair removal to prevent the regeneration of micro-vessels and recovery of the hair follicle, in conjunction with melasma treatment; in conjunction with IPL/laser and other treatments for dry eye; and in combination with treatment for psoriasis, or Sturge-Weber syndrome.

As non-limiting examples inflammatory skin diseases and other skin conditions include but are not limited to: rosacea, acne, atopic dermatitis, contact dermatitis, drug eruptions, psoriasis, seborrheic dermatitis, connective tissue diseases, autoimmune disorders, urticaria or hives, photodamage, aging, sunburn, skin infections, radiation dermatitis, skin exposed to ionizing radiation, port-wine stain birthmarks, hemangiomas, wounds or any injury, cherry angiomas, nevus araneuses, or skin or deeper tissues, acute or chronic wounds, photodamage, aging, sunburn, skin infections, radiation dermatitis, skin exposed to ionizing radiation.

As vasculature plays an important role in inflammation, and virtually any cytokine that affects blood vessel growth will also have an effect to modulate and regulate inflammation, rather than decrease or increase inflammation.

### Anti-VEGF Agents

The human VEGF-A gene is organized in eight exons. Alternative exon splicing results in the generation of four main VEGF isoforms, having, respectively, 121, 165, 189, and 206 amino acids following signal sequence cleavage (VEGF121, VEGF165, VEGF189, and VEGF206). VEGF165 is believed to be the most physiologically relevant isoform. For a review, see Ferrara et al., Biochem. Biophys. Res. Commun., 2005, 333, 328-335. The amino acid sequences of VEGF-A are well known in the art, and due to splice variation, the sequences are numerous. By way of non-limiting example, the following are exemplary and non-limiting GenBank®Accession Nos. for human VEGF-A (“VEGF”) amino acid sequences: AAP86646.1, P15692.2, NP_001191313.1, NP_001165101.1, NP_001165099.1, NP_001165097.1, NP_001165095.1, NP_001020539.2, NP_003367.4, NP_001165093.1, NP_001020541.2, NP_001191314.1, NP_001165100.1, NP_001165098.1, NP_001165096.1, NP_001165094.1, NP_001028928.1, NP_001020540.2, NP_001020538.2, and NP_001020537.2.

There are two VEGF receptor (VEGFR) tyrosine kinases (RTKs), Flt-1, known also as VEGFR-1 and KDR, Flk-1, or VEGFR-2. There is now agreement that VEGFR-2 is the major mediator of the mitogenic, angiogenic, and permeability-enhancing effects of VEGF. For a detailed review of the biological and signaling properties of the VEGFR, see Ferrara, Endocr. Rev. 2004, 25, 581-611. The amino acid sequences for the VEGFR are known in the art. By way of non-limiting example, GenBank®accession numbers for VEGFR-A amino acid sequences include (but are not limited to): NP_001153503.1, NP_002010.2, NP_001153502.1, and NP_001153392.1. The amino acid sequences for the VEGFR-2 are known in the art. By way of non-limiting example, GenBank®accession numbers for VEGFR-2 amino acid sequences include (but are not limited to): NP_002244.1, AAC16450.1, and NP_001153503.1.

Disclosed herein are methods for treating various skin conditions by administering an anti-VEGF agent to a subject. In one embodiment, the anti-VEGF antibody bevacizumab can be used in the present methods. The antibody bevacizumab and its VEGF-binding activity are reviewed in detail in Ferrara et al., Biochem. Biophys. Res. Commun., 2005, 333, 328-335. Bevacizumab may be administered to skin (e.g., for the inhibition of keloid recurrence) at a dose about 5-15 mg. In one embodiment, the dose for administration to site of keloid removal (e.g., for inhibition of keloid recurrence) is about 10 mg).

It is to be appreciated, however, that the treatment method described herein can also be performed using other anti-VEGF agents (e.g., VEGF or VEGFR inhibitors, such as, but not limited to, other anti-VEGF antibodies, drugs, prodrugs, small molecules, peptides, nucleic acid inhibitors (e.g., siRNA, shRNA, antisense oligonucleotides), fusion proteins, etc.), either that are known in the art or that will be discovered or engineered in the future, so long as the anti-VEGF agent has the ability to inhibit the action of VEGF (e.g., human VEGF) and/or a VEGFR (e.g., VEGFR-1 and/or VEGFR-2)(e.g., human VEGFR-1 or human VEGFR-2)(i.e., to inhibit VEGF signaling). Assays for determining whether an antibody or other agent interferes with VEGF signaling (either by inhibiting VEGF or a VEGFR or the interaction between VEGF and its receptor), for example, are well known in the art, and can be used to determine whether an anti-VEGF agent interferes with VEGF signaling and is therefore encompassed by the presently disclosed methods. Non-limiting examples of such assays include the VEGF inhibition assays described in Vicari et al., J. Biol. Chem., 2011, 286(15), 13612-25 and Brekken et al. Cancer Res., 2000, 60, 5117-24.

By way of non-limiting example, other anti-VEGF antibodies and inhibitors that are known in the art, and, that can be used in the methods disclosed herein include but are not limited to: ranibizumab, pegaptanib, imatinib, vandetanib, sorafenib, pazopanib, valatanib, vevasiranib, aflibercept, etanercept, anecortave acetate (angiostatic steroid), VEGF-trap (a fusion protein), squalamine lactate, edotinib, gefitinib (small molecules), Combretastatin A4 Prodrug (an antitubulin/antiangiogenic agent), AdPEDF (Adenovector pigment epithelium-derived factor), Cand5 (siRNA), protein tyrosine kinase 7 inhibitors (PTK7), lipolytic agents, TG100801, AGO13958, AL39324, AGN211745 (VEGF receptor blockers), anti-angiogenic VEGF-A(xxx)b family, VEGF Trap (receptor decoy), protein kinase antibodies to tyrosine kinase inhibitor receptors SIM010603, kinase domain receptor antibodies (KDR1.3 and KDR2.6), GS101 aganirsen (an antisense oligonucleotide against insulin receptor substrate aka IRS-1), picropodophyllin (PPP), tetrameric tripeptide, tissue kallikrein, KH906 (a recombinant human VEGF receptor protein fusion), beta-adreno receptor blocker.beta.3-AR, nicotinic acetycholine receptor antagonists, linomide analogue (LinOS), morpholino oligomers (VEGFR1_MOel3), decursin, prorenin, vasohibin and sirolimus. It will be appreciated that because the amino acids sequences (as well as nucleic acid sequences encoding the amino acid sequences) of VEGF and VEGFRs are known in the art, the skilled artisan can readily design additional anti-VEGF agents for use in the presently disclosed methods.

Dosage ranges for anti-VEGF agents, e.g., those disclosed above, can be readily determined by the ordinarily skilled artisan, and can, e.g., first be determined in animal models for determining dosage, safety and efficacy according to standard methods known in the art.

### Anti-Inflammatory Steroids

Anti-inflammatory steroids are steroidal compounds that have anti-inflammatory activity and include corticosteroids, including glucocorticoids. Glucocorticoids bind to glucocorticoid receptors in the cytoplasm which may increase the transcription of genes coding for anti-inflammatory proteins, including lipocortin-1, interleukin-10, interleukin-1 receptor antagonist and neutral endopeptidase. Glucocorticoids also inhibit the expression of multiple inflammatory genes, including genes for various cytokines, enzymes, receptors and adhesion molecules. Barnes et al., Clin. Sci., 1998, 94, 557-572.

Suitable steroids for application to the skin include, e.g., alclometasone 0.05% cream (generic or Aclovate®0.05% cream available from PharmaDerm Inc.), diflorasone 0.005% cream, prednicarbate 0.1% (generic, or Dermatop 0.1% cream or ointment available from Sanofi-Aventis US LLC), and fluocinonide cream 0.1% (e.g., Vanos®, available from Medicis Inc.). Suitable dosages for administration to humans include, e.g., 0.05% to 0.1%. Further commercially available topical ocular steroids suitable for use in the methods described herein are listed in Table 3.

TABLE 3
Commercially Available Topical Steroids

| Potency | Drug |
|---|---|
| Super | Betamethasone dipropionate, augmented; Clobetasol |
| High | propionate; Fluocinonide; Flurandrenolide; Halobetasol |
| | propionate |
| High | Amcinonide; Betamethasone dipropionate; Desoximetasone; |
| | Diflorasone diacetate; Fluocinonide; Halocinonide; |
| | Triamcinolone |
| Medium- | Amcinonide; Betamethasone dipropionate; Betamethasone |
| High | valerate; Desoximetasone; Diflorasone diacetate; |
| | Fluocinonide; Fluticasone propionate; Mometasone furoate; |
| | Triamcinolone acetonide |
| Medium | Clocortolone pivalate; Fluocinolone acetonide; Hydrocortisone |
| | valerate; Mometasone furoate; Triamcinolone acetonide |
| Medium- | Betamethasone valerate; Desonide; Fluocinolone acetonide; |
| Low | Fluticasone proprionate; Hydrocortisone butyrate; |
| | Hydrocortisone probutate; Hydrocortisone valerate; |
| | Prednicarbate; Triamcinolone acetonide |
| Low | Aclometasone dipropionate; Betamethasone valerate; |
| | Desonide; Fluocinolone acetonide; Triamcinolone acetonide |
| Very Low | Hydrocortisone (base); Hydrocortisone acetate/Aloe vera; |
| | Hydrocortisone acetate/urea |

### Non-Steroidal Anti-Inflammatory Drugs (NSAIDs)

Non-steroidal anti-inflammatory drugs (NSAIDs) are non-steroidal compounds that reduce inflammation. Most NSAIDs act as nonselective inhibitors of the enzyme cyclooxygenase (COX), inhibiting both the cyclooxygenase-1 (COX-1) and cyclooxygenase-2 (COX-2) isoenzymes. COX catalyzes the formation of prostaglandins. Since COX-1 inhibition is believed to be associated with gastrointestinal side-effects of NSAIDs, compounds that are selective COX-2 inhibitors have also been developed.

Non-limiting examples of NSAIDs that can be administered to the skin include, e.g., diclofenac 1% (topical) (e.g., Voltaren gel 1% from Novartis Pharmaceuticals Corp. or Pennsaid 1.5% solution (topical) from Mallinckrodt Pharmaceuticals). Suitable dosages for administration to humans include, e.g., 1% (topical) apply to skin once a day to 40 mg (administer by injecting 1 mL into skin).

### Methods of Administering/Applying Anti-VEGF Compounds Transdermally

Transdermal delivery systems, such as transdermal patches, are described as one system or device for the administration and delivery of the AVEGF comprising compounds described herein. One benefit of therapeutically treating skin transdermally is that it avoids metabolization of the pharmaceutically active agent in the liver, which may occur upon oral administration of the described AVEGF compounds. Accordingly, administering the described AVEGF compounds through a transdermal delivery system avoids unwanted side effects in the liver and gastrointestinal. Additionally, transdermal administration of pharmaceutically active agents usually requires less of the pharmaceutically active agent to have the same effect. Furthermore, testing has shown that the transdermal delivery systems described herein provide a more constant blood level of the pharmaceutically active agent as the agent is immediately effective in a systemic manner upon permeation through the skin.

Topically via trans-dermal patch, lipid-based nanocarriers, such liposomes and micelles, solvents that enable transit across the skin barrier. In one embodiment, the transdermal patch may include dimethyl sulfoxide, which is shown to be an effective penetration enhancer for topical administration of NSAIDs.

In an embodiment, AVEGF may be included in a matrix release permeable patch in a compound such as hyaluronic acid allowing slow release into the target area. The AVEGF patch would be designed to be used specifically for treating the skin conditions described herein.

In one embodiment AVEGF would be applied via a transdermal patch to target areas subsequent to dermabrasion or ablative fractional resurfacing, or micro-needling. The described AVEGF could be compounded with other aids for treating skin conditions such as hyaluronic acid, vitamin C, hydroquinone, corticosteroids, tretinoin.

In addition, in an embodiment, the AVEGF could be compounded with at least one anti-inflammatory steroid, at least one non-steroid anti-inflammatory drug, or combinations of any of these components.

### Various Transdermal Delivery Systems

### A. Patches

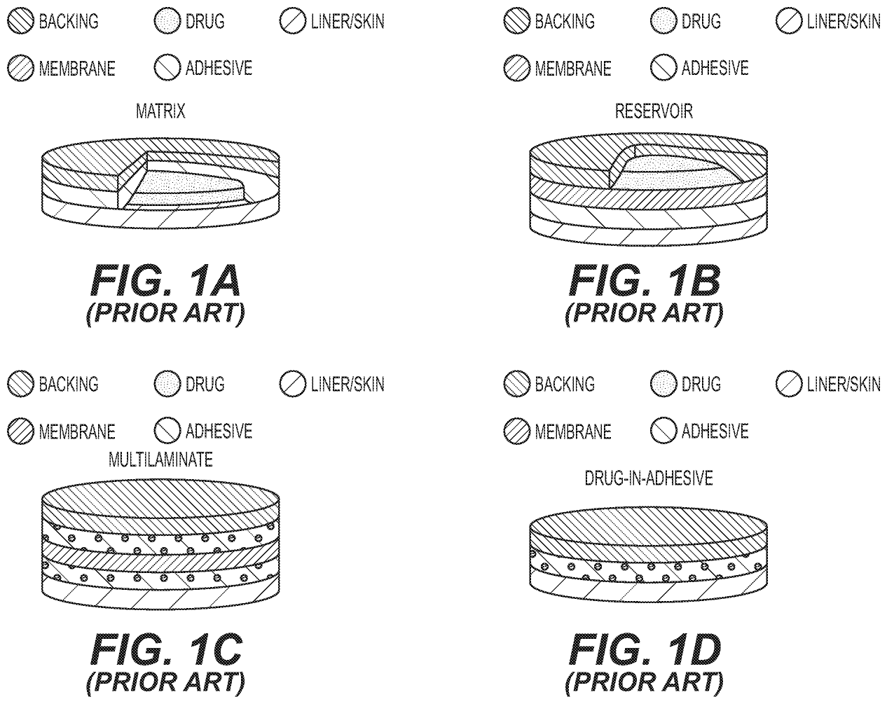

The AVEGF compound described herein may be contained in a transdermal patch. Typically, transdermal patches comprise six elements. Depending on the exact design, not all elements are required. With reference to FIGS. 1A-1D, in an embodiment, a transdermal patch described herein may take a variety of forms and comprise various layers and locations for the active ingredients. For example, in an embodiment, a patch may comprise an outermost layer, which protects the formulation during the period when the patch is located on the skin. The patch may also comprise a membrane, which controls the rate of the drug release out of the patch and into the skin. The patch described herein also contains an adhesive, which is located in the skin contacting layer that adheres the patch to the skin. In another embodiment, the adhesive further includes the AVEGF containing compound. The patch described herein may also contain an overlaminate tape. This is an external protective covering or functional layer which can be directly integrated into the patch design. Finally, the patch described herein may also contain a release liner, which protects the skin-contacting adhesive during storage and is removed prior to application of the patch.

Depending on the different uses and the desired therapeutic treatment, the transdermal patches described herein can be designed in multiple ways. The different designs are influenced by a variety of factors, including the properties of the drug, the dosage level, the treatment area and the time required to administer the drug.

Again with reference to FIGS. 1A-1D, in one embodiment, the transdermal patch comprises a matrix or drug in adhesive design. FIG. 1D. This design blends the AVEGF, typically compounded with one or more of the elements described above, directly into the adhesive of the patch. This is the most common type of patch, as is referred to as the drug-in adhesive, or DIA. FIG. 1D.

In another embodiment, the transdermal patch described herein comprises a classic reservoir design. FIG. 1B. In this embodiment, the AVEGF containing compound is located in a blister pouch, which has a rate-controlling membrane on one side and impervious backing on the other.

In another embodiment, the transdermal patch described herein comprises a polymer reservoir design. FIG. 1A. In this embodiment, the AVEGF containing compound is located in a semi-solid drug containing polymer matrix, which is in direct contact with the skin, with an adhesive ring around the matrix to adhere to the skin.

In another embodiment, the transdermal patch described herein comprises a multi-laminate solid-state seservoir design. FIG. 1C. In this embodiment, the AVEGF containing compound is similar to DIA design (FIG. 1D), with multiple layers comprising the AVEGF, typically compounded with one or more of the elements described above, with the AVEGF directly in the adhesive of the patch, where each layer is separated by a membrane. This design allows for the delivery of two drugs at different release times. This may be used, for example, when a bolus dose is needed to begin treatment, which is followed by a maintenance dose of the therapeutically active compound.

In one embodiment AVEGF is integrated into a bandage strip specifically designed for areas of the body that have been exposed to a skin treatment or surgical incision, as described herein. The surgical strip would be permeable to promote healing and would contain a concentration of AVEGF in an ointment that would moderate the transfer into the treated area, such as the surgical incision during healing. AVEGF strips would be supplied to the patient to reapply one or more times per day, or for one to 100 days to maintain the desired concentration of AVEGF. The design of the strips would be specific for use with linear incisions such as incisions created during face lifts, chin implants, blepharoplasty, breast implants, cosmetic procedures, and incisions from medical procedures such as sternotomy that has a high incident of hypertrophic scarring. AVEGF surgical strips would have a predetermined concentration of AVEGF in a media and the strips would be specifically designed to be consumable and reapplied by the patient.

In one embodiment a permeable bandage strip with a predetermined concentration of AVEGF in a gel or ointment and an adhesive region surrounding the region of the AVEGF would be packaged as an aid to prevent or reduce keloid and scar formation after surgery or injury. The AVEGF bandage would be designed to be packages for application by a patient.

In one embodiment a permeable bandage strip with a predetermined concentration of AVEGF in a gel or ointment and an adhesive region surrounding the region of the AVEGF would be packaged as an aid to prevent or reduce keloid and scar formation after Mohs surgery or mole removal. The concentration of AVEGF would be predetermined to prevent scars and as a precaution for precancerous vessel growth. The AVEGF bandage would be designed to be packaged for application by a patient to the region where the mole removal or Mohs surgery.

In an embodiment, the patch or bandage strip may be in the form of a sheet or roll. The sheet or roll may be pre-cut or perforated, so as to comprise patches of pre-cut sizes and shapes. For example, the pre-cut patch has a circular shape with a diameter ranging from 1 mm to 80 mm, such as from 2 mm to 60 mm, or even 5 mm to 50 mm. In an embodiment, the pre-cut patch may have an oval shape with the major diameter ranging from 5 mm to 100 mm, such as 10 mm to 80 mm, or even 15 mm to 60 mm.

In another embodiment, the patch or bandage strip may be in the form of a sheet or roll that is not pre-cut or perforated. Rather, in this embodiment, the patch or bandage strip is in the form of a sheet or tape that can be cut into desired shapes and sizes, such as by a distributor or the end user.

In one embodiment AVEGF is incorporated into a disc pad less than 1 inch in diameter and preferably about 0.5″ in diameter to be used in ear piercing, and body piercing for patients susceptible to keloid formation or other scarring. The pad is specifically designed to be used in conjunction with body piercing as a disposable pad.

In this embodiment VEGF can be administered trans-dermally via body paint in a latex or water-soluble solution. AVEGF paint can be self-administered by a patient to an affected area such as an area of skin subject to keloid or scar formation. The advantage over a patch is that the treatment can be easily confined to the target area with regular doses to maintain predetermined concentration levels in the target area. Patches overlapping non-target areas will release AVEGF to non-target areas increasing cost of dosage, and also result is systemic effects. Patches also lose transfer rates as concentration drops. An occlusive paint such as latex or other lipid, oil, or non-water-soluble compound can be peeled off and reapplied as concentration levels drop.

In one embodiment topical and mechanical methods increasing or enabling skin penetration including a combination of fractional ablative treatment, micro-needling and the like provide a porous tissue surface for enhanced penetration of AVEGF.

In one embodiment methods and systems enhance the penetration of AVEGF solutions by thinning or removing the stratum corneum with lasers or other methods. dermabrasion, RF surface ablation, plasma resurfacing, laser micropeel, fractional laser ablation

In conjunction with a device such as micro-needling with and without RF, lasers-both fractionated and non-fractionated, after laser micropeel or dermabrasion, enhanced penetration with ultrasound, with heat.

### Methods and Devices to Ensure Slow, Sustained or Timed Release of Anti-VEGF Compounds

As a non-limiting example anti-VEGF containing compounds are utilized as fillers and the like, by combining them with fillers such as Hyaluronic Acid fillers, collagen, elastin, proteoglycans such as decorin, versican, chondroitin sulfate, herparin sulphate proteoglycan, elastin, fibrillin, fibulin (refer to abandon filler patent attached), collagens (all types) etc.

In one embodiment sustained-release of anti-VEGF compounds are provided, including but not limited to: carrier molecules; extra-cellular matrix molecules; synthetic compounds and the like.

In one embodiment sustained or timed release of anti-VEGF compounds are provided that comprise albumin and similar carrier proteins. In an embodiment, the sustained or timed release of anti-VEGF compounds comprise the disclosed AVEGF with at least one corticosteroids, hydroquinone and other drugs.

### Methods and Devices Utilizing Sensors Monitoring the Target Area

In an embodiment, there is described an algorithm to determine an amount of AVEGF to apply, and a means to apply the AVEGF such that the AVEGF is applied over a time period and at a concentration or level that provides more efficient use.

In one embodiment an oxygen sensor is used to monitor the oxygen level in the target tissue, and an algorithm that uses the oxygen level to determine the perfusion of blood into tissue and adjusts the application of AVEGF so that angiogenesis is controlled. Near infrared spectroscopy can be used to measure tissue oxygen level non-invasively for skin surface applications. In one embodiment, an optical fiber can be used to monitor deeper injuries, conditions, tumors, or such target areas where angiogenesis is to be monitored.

In an embodiment, pulse oximetry can be used to determine the oxygen level in tissue over extended areas by distributing the pulse oximeters in an array. An array of pulse oximeters utilizing red and infrared light overlapping an array of detectors can be used. The light or signal array can be an array of LEDs, a light source coupled to optical fibers or a holographic lens. The detectors can be an array of solid state detectors or an array of light collectors such as optical fibers and a single or multiple detectors. In one embodiment there is a red and infrared source coupled through a microlens array or array of optical fibers and an array of optical fibers that collect the light reflecting or passing through the target tissue and transmitting it to a detector that averages the pulse oximetry signal over the target area. An algorithm determines the amount of AVEGF to apply, the AVEGF is applied to the area of tissue feeding the blood supply of the target area.

In an embodiment, a reducing agent such as calcium, oxalic acid, ascorbic acid, carbon monoxide can be used to lower the oxygen level in the target tissue to a level not beneficial to angiogenesis or to a level to slow angiogenesis. For CO, applications in solution of less than 100 ppm, in most cases less than 35 ppm directly applied to target tissue will cause the formation of carboxyhemoglobin lowering or preventing 02 in the target tissue lower or preventing angiogenesis or tissue growth. A CO-oximeter can be used in conjunction with a pulse oximeter to determine a concentration of carboxyhemoglobin sufficient to reduce angiogenesis, scar development, keloids or tumors in the target region. CO is readily eliminated by the body so that small micro-concentrations of less than 35 ppm will be confined to the target area. I found no use of CO in localized micro-concentrations for treating any diseases. Levels of 26 ppm for 1 hour are considered safe, 9 ppm for 8 hours. CO level rapidly drops outside of the applied area so intermittent applications can stop angiogenesis, neurogenesis, keloid formation, tumor growth in a very localized area at levels that drop safely in surrounding tissue for predetermined safe times. Thus, CO can be generated in very low concentrations and used safely in localized areas analogous to botulism toxin, Botox, an extremely toxic poison can be used locally in very small local concentrations.

In an embodiment, the device for monitoring CO, 02 or other indicators of scar, keloid, angiogenesis, determining levels of medication, and administering medication can be worn on a belt or attached to a patient such that the device can efficiently apply AVEGF, compounds of AVEGF, or other anti-angiogenesis solutions at predetermined concentrations for predetermined time periods.

In an embodiment, the sensor can be monitored wirelessly.

In Combination with Stem Cell Therapy, Growth Factors, and PRP Specifically to Prevent Stem Cell Stimulated Angiogenesis.

In one embodiment methods and systems are providing for the effects of AVEGF in conjunction with stem cell therapy and platelet-rich-plasma (PRP) to control angiogenesis. In one embodiment methods and devices directly administer AVEGF with multi-needle injectors, precision injectors, air injectors.

In one embodiment devices and systems are provided whereby AVEGF would be added to PRP in a concentration that would affect angiogenesis to a desired level. Applying PRP into damaged tissues will stimulate body growth of new, healthy cells and promote healing because the tissue growth factors are more concentrated in the prepared growth injections however PRP is not selective and can promote angiogenesis to an undesired level creating prolonged redness and scarring. The device includes predetermined amounts of AVEGF that are added to and are stable in the patient's PRP. The device includes a means of adding AVEGF to PRP in a closed sterile manner such that it can be safely applied or injected into damaged or treated tissue.

In one embodiment the device for adding AVEGF to PRP would be a sterile kit that includes predetermined amounts of AVEGF. In another embodiment the kit could be sterilized by autoclave, soaking or other methods and include a means for adding predetermined amounts of AVEGF. In one embodiment the PRP is separated into a means for injecting into tissue such mean includes a predetermined amount of AVEGF.

### Methods and Devices are Provided for the Controlled Release Drug Delivery of AVEGF Solutions Including but not Limited to Micro-Encapsulation and the Like

In an embodiment, the described AVEGF compounds may be encapsulated in liposomes that can be triggered by light, lasers, ultrasound, or decay over time AVEGF. Alternatively, the described AVEGF compounds may be microencapsulated in a polymer carrier, which can then be injected into the target tissue. In this embodiment, the AVEGF can be released by applying ultrasound at sufficient levels to release the microencapsulated AVEGF. In this embodiment, the ultrasound may be applied in a fractional pattern by high intensity focused ultrasonic transducer (HIFU) to release a fraction of the AVEGF and subsequent treatments can release AVEGF non-invasively at predetermined intervals by treating a fractionally array in the target area, for example a grid of treated areas making up a predetermined percentage of the target area.

In one embodiment, there is described a non-invasive method of treating a subject at predetermined intervals by using an AVEGF containing compound that is microencapsulated in a polymer microcapsule carrier and injected into the target tissue. In this embodiment, the AVEGF is released by applying ultrasound at sufficient levels to release the microencapsulated AVEGF the ultrasound may be applied in a fractional pattern by high intensity focused ultrasonic transducer to release a fraction of the AVEGF and subsequent treatments can release AVEGF non-invasively at predetermined intervals.

There is also described the use of colloidal or polymeric capsule for micro-encapsulation and nano-encapsulation of AVEGF solutions for controlled release of AVEGF and injectors for administering micro-encapsulated AVEGF solutions.

### Photo-Thermally Triggered Delivery

In one embodiment, there is described a method of photothermally triggering delivery of the anti-VEGF compounds described herein using various inorganic nanoparticles. As used herein, nanoparticles are intended to mean particles generally ranging in dimensions from 1 nm to a few hundred nanometers in at least one dimension. The nanoparticles described herein are designed to be used with or carry anti-VEGF compounds. In one embodiment, the nanoparticles may comprise gold, silver and iron-oxide, which possess desired photothermal properties. In one embodiment, there is described anti-VEGF compounds comprising gold and silver nanoparticle to mediate inhibition of angiogenesis. In one embodiment, there is described anti-VEGF compound including gold nanoparticles to photothermally control the release of angiogenesis inhibiting agents with photo or radio wave activation.

In one embodiment, there is described metal nanoparticles, such as gold nanoparticles, conjugated with an anti-angiogenic peptide which can be combined with visible laser irradiation to enhance angiogenesis arrest in vivo. The combination of a green laser coupled to gold nanoparticles can achieve high localized temperatures able to precisely cauterize blood vessels, that when combined with VEGF pathway inhibition, such as the transdermal application of anti-VEGF, can reduce FLT-1 expression.

In one embodiment, there is described a method of photothermally triggering delivery with laser light. In one non-limiting embodiment, a 532 nm laser may be used in conjunction with gold nanoparticles that have been conjugated with anti-angiogenic peptides. It has been discovered that the laser causes a significantly higher increase in temperature for long term low intensity exposure. This has the effect of increasing activity and at the same time coagulating vessels. In another embodiment, there is described RF or light interaction with other nanoparticles described herein. This has been shown to enhance activity by heating, or to release conjugated anti-angiogenesis compounds from nanoparticles with thermal shock or pulsed energy.

### Methods and Devices of Anti-VEGF Embedded Implants

In one embodiment AVEGF is integrated onto the surface of implants for the body to allow the above described benefits of AVEGF to be slowly released at the internal surgical site. For example, there is described a silicone breast implant having an AVEGF compound described herein integrated on the surface in a slow release polymer or lipid film to prevent capsular contracture. The implant is designed with a biocompatible layer containing a predetermined concentration of AVEGF in a HA, or other media.

In one embodiment AVEGF is incorporated onto the surface of surgical implants such as chin implants, face implants to help prevent and reduce the formation of scars. The implant is designed with a biocompatible layer containing a predetermined concentration of AVEGF in a hyaluronic acid, or other media.

In one embodiment, there is described a suture thread saturated with an AVEGF compound as described herein that would remediate the development of inflammation and angiogenesis at the suture needle sites reducing the development of scar formation.

In one embodiment AVEGF is incorporated into stents to prevent restenosis, whereas the stent is coated with a polymeric-like compound that contains anti-VEGF that is released over a time period sufficient to prevent restenosis.

### Devices Such as Jet-Injectors, Needles Including Arrays of Microneedle Injectors, and Atomized Sprayers

A device configured to deliver AVEGF into target tissue at predetermined depths. Lower concentrations of AVEGF can be used if applied directly to the area of interest rather than migration through a concentration gradient in tissue. In one embodiment AVEGF is applied by microneedles over a predetermine area and over a predetermined range of depths where the area predominantly matches the area of the target tissue and the range of depths extends mostly through the entire depth of the target tissue whereby the target tissue is an area subject to scar or keloid formation.

A device with multiple needles spaced such that anti-VEGF solution is dispersed completely into the target area such as an area predisposed to developing keloids, or a surgical scar.

### Different Inflammatory Skin Diseases to be Treated

The foregoing delivery mechanism can be used to treat a variety of skin conditions, such as those described above, as well as the following different and non-limiting inflammatory skin diseases. Each of the following diseases/conditions is either primarily inflammatory or has inflammation that at least partially cause symptoms of the diseaselcondition. Thus, the systems, devices and methods disclosed herein will be suitable for treating the following diseases/conditions:

Acneiform eruptions. In one embodiment, there is provided systems, devices and methods for treating Acneiform eruptions are caused by changes in the pilosebaceous unit.

Autoinflammatory syndromes. In one embodiment, there is provided systems, devices and methods for treating autoinflammatory syndromes. Autoinflammatory syndromes are a group of inherited disorders characterized by bouts of inflammatory skin lesions and periodic fevers.

Chronic Blistering. In one embodiment, there is provided systems, devices and methods for treating chronic blistering. Chronic blistering cutaneous conditions have a prolonged course and present with vesicles and bullae.

Mucous Membranes. In one embodiment, there is provided systems, devices and methods for treating conditions of the mucous membranes including conditions of the moist linings of the eyes, nose, mouth, genitals, and anus.

Glands. In one embodiment, there is provided systems, devices and methods for treating conditions of the skin appendages that are those affecting the glands of the skin, hair, nails, and arrector pili muscles.

Subcutaneous fat. In one embodiment, there is provided systems, devices and methods for treating conditions of the subcutaneous fat. Conditions of the subcutaneous fat are those affecting the layer of adipose tissue that lies between the dermis and underlying fascia.

Congenital Anomalies. In one embodiment, there is provided systems, devices and methods for treating congenital anomalies. Cutaneous congenital anomalies are a diverse group of disorders that result from faulty morphogenesis, the biological process that forms the shape of a human body.

Connective Tissue Diseases. In one embodiment, there is provided systems, devices and methods for treating connective tissue diseases. Connective tissue diseases are caused by a complex array of autoimmune responses that target or affect collagen or ground substance.

Dermal Fibrous and Elastic Tissue. In one embodiment, there is provided systems, devices and methods for treating abnormalities of dermal fibrous and elastic tissue. Abnormalities of dermal fibrous and elastic tissue are caused by problems in the regulation of collagen synthesis or degradation.

Dermal and Subcutaneous Growths. In one embodiment, there is provided systems, devices and methods for treating dermal and subcutaneous growths. Dermal and subcutaneous growths result from (1) reactive or neoplastic proliferation of cellular components of the dermis or subcutaneous tissue, or (2) neoplasms invading or aberrantly present in the dermis.

Dermatitis. In one embodiment, there is provided systems, devices and methods for treating various types of Dermatitis. Dermatitis is a general term for “inflammation of the skin” as includes but is not limited to childhood granulomatous periorificial dermatitis and Essential dermatitis. Also included in this definition is atopic dermatitis, which is defined as a chronic dermatitis associated with a hereditary tendency to develop allergies to food and inhalant substances. Non-limiting examples of Atopic dermatitis includes atopic eczema, disseminated neurodermatitis, flexural eczema, infantile eczema, prurigo diathsique. Also included within this definition is contact dermatitis, which is defined as dermatitis caused by certain substances coming in contact with the skin.

Eczema. In one embodiment, there is provided systems, devices and methods for treating eczema. Eczema refers to a broad range of conditions that begin as spongiotic dermatitis and may progress to a lichenified stage.

Pustular dermatitis. In one embodiment, there is provided systems, devices and methods for treating pustular dermatitis. Pustular dermatitis is an inflammation of the skin that presents with pustular lesions. Non-limiting examples include: Eosinophilic pustular folliculitis (Ofuji's disease, sterile eosinophilic pustulosis); Reactive arthritis (formerly known as Reiter's syndrome); and Subcorneal pustular dermatosis (Sneddon-Wilkinson disease).

Seborrheic Dermatitis. In one embodiment systems, devices and methods are provided for treating seborrheic dermatitis. Seborrheic dermatitis is a chronic, superficial, inflammatory disease characterized by scaling on an erythematous base.

Pigmentation Disturbances. In one embodiment, there is provided systems, devices and methods for treating disturbances of human pigmentation. Disturbances of human pigmentation, either loss or reduction, may be related to loss of melanocytes or the inability of melanocytes to produce melanin or transport melanosomes correctly.

Drug Eruptions. In one embodiment, there is provided systems, devices and methods for treating Drug eruptions, which are defined as adverse drug reactions that present with cutaneous manifestations. Non-limiting examples include: Adverse reactions to biologic agents and cytokines; Chemotherapy-induced acral erythema (palmoplantar erythrodysesthesia syndrome) and hyperpigmentation; Drug-induced acne, angioedema, gingival hyperplasia, lupus erythematosus, nail changes, pigmentation and the like; Injection site reaction; Vitamin K reaction; and Warfarin necrosis

Endocrine Conditions. In one embodiment, there is provided systems, devices and methods for treating endocrine conditions, which often present with cutaneous findings as the skin interacts with the endocrine system in many ways.

Eosinophilic Cutaneous Conditions. In one embodiment, there is provided systems, devices and methods for treating eosinophilic cutaneous conditions, which encompass a wide variety of diseases that are characterized histologically by the presence of eosinophils in the inflammatory infiltrate, or evidence of eosinophil degranulation.

Epidermal nevi, neoplasms, and cysts. In one embodiment, there is provided systems, devices and methods for treating epidermal nevi, neoplasms, and cysts are skin lesions that develop from the epidermal layer of the skin.

Erythemas. In one embodiment, there is provided systems, devices and methods for treating erythemas are reactive skin conditions in which there is blanchable redness.

Genodermatoses. In one embodiment, there is provided systems, devices and methods for treating Genodermatoses, which are inherited genetic skin conditions often grouped into three categories: chromosomal, single gene, and polygenetic.

Infection-Related Conditions. In one embodiment, there is provided systems, devices and methods for treating infection-related cutaneous conditions may be caused by bacteria, fungi, yeast, viruses, or parasites.

Bacterium-related. In one embodiment, there is provided systems, devices and methods for treating bacterium-related cutaneous conditions often have distinct morphologic characteristics that may be an indication of a generalized systemic process or simply an isolated superficial infection.

Mycobacterium-related. In one embodiment, there is provided systems, devices and methods for treating mycobacterium-related cutaneous conditions are caused by mycobacterium infections.

Mycosis-related. In one embodiment, there is provided systems, devices and methods for treating mycosis-related cutaneous conditions caused by fungi or yeasts, and may present as either a superficial or deep infection of the skin, hair, or nails.

Parasitic infestations, stings, and bites. In one embodiment, there is provided systems, devices and methods for treating parasitic infestations, stings, and bites in humans caused by several groups of organisms belonging to various forms of animals and insects, including the following phyla: Annelida, Arthropoda, Bryozoa, Chordata, Cnidaria, Cyanobacteria, Echinodermata, Nemathelminthes, Platyhelminthes, and Protozoa.

Virus-related. In one embodiment, there is provided systems, devices and methods for treating virus-related cutaneous conditions, which are caused by two main groups of viruses-DNA and RNA types-both of which are obligatory intracellular parasites.

Lichenoid eruptions. In one embodiment, there is provided systems, devices and methods for treating lichenoid eruptions, which are dermatoses related to the unique, common inflammatory disorder lichen planus, which affects the skin, mucous membranes, nails, and hair.

Lymphoid-related. In one embodiment, there is provided systems, devices and methods for treating lymphoid-related cutaneous conditions, which are a group of disorders characterized by collections of lymphocyte cells within the skin.

Melanocytic nevi and neoplasms. In one embodiment, there is provided systems, devices and methods for treating melanocytic nevi and neoplasms, which are caused by either a proliferation of (1) melanocytes, or (2) nevus cells, a form of melanocyte that lack dendritic processes.

Melanoma. In one embodiment, there is provided systems, devices and methods for treating melanoma, which is a malignant proliferation of melanocytes and the most aggressive type of skin cancer.

Monocyte- and macrophage-relate. In one embodiment, there is provided systems, devices and methods for treating monocyte- and macrophage-related cutaneous conditions, which are characterized histologically by infiltration of the skin by monocyte or macrophage cells, often divided into several categories, including granulomatous disease, histiocytoses, and sarcoidosis.

Mucinoses. In one embodiment, there is provided systems, devices and methods for treating mucinoses, which are classified as a group of conditions caused by dermal fibroblasts producing abnormally large amounts of mucopolysaccharides.

Neurocutaneous. In one embodiment, there is provided systems, devices and methods for treating neurocutaneous conditions, which are conditions that occur due organic nervous system disease or are psychiatric in etiology.

Noninfectious immunodeficiency-related. In one embodiment, there is provided systems, devices and methods for treating noninfectious immunodeficiency-related cutaneous conditions are caused by T-cell or B-cell dysfunction.

Nutrition-related. In one embodiment, there is provided systems, devices and methods for treating nutrition-related cutaneous conditions are caused by malnutrition due to an improper or inadequate diet. Non-limiting examples include: Biotin deficiency; Carotenemia; Essential fatty acid deficiency; Folic acid deficiency; Hypervitaminosis A; Hypovitaminosis A (phrynoderma); Iron deficiency; Kwashiorkor; Lycopenemia; Maple syrup urine disease; Marasmus; Niacin deficiency (pellagra, vitamin B₃ deficiency); Selenium deficiency; Vitamin B₁ deficiency (beriberi, thiamine deficiency); Vitamin B₁₂ deficiency (cyanocobalamin deficiency); Vitamin B₂ deficiency (ariboflavinosis, riboflavin deficiency); Vitamin B₆ deficiency (pyridoxine deficiency); Vitamin B₆ excess (pyridoxine excess); Vitamin C deficiency (scurvy); Vitamin K deficiency; and Zinc deficiency.

Papulosquamous hyperkeratotic. In one embodiment, there is provided systems, devices and methods for treating papulosquamous hyperkeratotic cutaneous conditions are those that present with papules and scales caused by a thickening of the stratum corneum.

Palmoplantar keratodermas. In one embodiment, there is provided systems, devices and methods for treating palmoplantar keratodermas, which are a diverse group of hereditary and acquired keratodermas in which there is hyperkeratosis of the skin of the palms and soles.

Pregnancy-related. In one embodiment, there is provided systems, devices and methods for treating pregnancy-related cutaneous conditions are a group of skin changes observed during pregnancy.

Pruritic. In one embodiment, there is provided systems, devices and methods for treating pruritus, which is commonly known as itchiness. This condition is a sensation exclusive to the skin, and characteristic of many skin conditions.

Psoriasis. In one embodiment, there is provided systems, devices and methods for treating psoriasis, which is a common, chronic, and recurrent inflammatory disease of the skin characterized by circumscribed, erythematous, dry, scaling plaques.

Reactive neutrophilic. In one embodiment, there is provided systems, devices and methods for treating reactive neutrophilic cutaneous conditions. These conditions constitute a spectrum of disease mediated by neutrophils, and typically associated with underlying diseases, such as inflammatory bowel disease and hematologicmalignancy.

Recalcitrant palmoplantar eruptions. In one embodiment, there is provided systems, devices and methods for treating recalcitrant palmoplantar eruptions, which are skin conditions of the palms and soles that are resistant to treatment.

Resulting from errors in metabolism. In one embodiment, there is provided systems, devices and methods for treating skin conditions resulting from errors in metabolism are caused by enzymatic defects that lead to an accumulation or deficiency of various cellular components, including, but not limited to, amino acids, carbohydrates, and lipids.

Resulting from physical factors. In one embodiment, there is provided systems, devices and methods for treating skin conditions resulting from physical factors occur from a number of causes, including, but not limited to, hot and cold temperatures, friction, and moisture. Non-limiting examples include: Abrasion; Callus (callosity, clavus, corn, heloma, heloma durum, heloma molle, intractable plantar keratosis, tyloma); Friction blister, Frostbite; Jogger's nipple; Photoaging (dermatoheliosis), Sunburn; Thermal burn; Turf toe, and Wrestler's ear (cauliflower ear, traumatic auricular hematoma).

Ionizing radiation-induced. In one embodiment, there is provided systems, devices and methods for treating ionizing radiation-induced cutaneous conditions. Non-limiting examples include conditions resulting from exposure to radiation therapies, such as for cancer.

Urticaria and angioedema. In one embodiment, there is provided systems, devices and methods for treating urticaria, which is a vascular reaction of the skin characterized by the appearance of wheals, which are firm, elevated swelling of the skin. Angioedema, which can occur alone or with urticaria, is characterized by a well-defined, edematous swelling that involves subcutaneous tissues, abdominal organs, or upper airway.

Vascular-related. In one embodiment, there is provided systems, devices and methods for treating vascular-related cutaneous conditions result from dysfunction of the blood or blood vessels in the dermis, or lymphatics in the subcutaneous tissues.

It will be apparent to those skilled in the art that various modifications and variations can be made to the disclosed alloy and method of forming the alloy into a finished part without departing from the scope of the disclosure. Alternative implementations will be apparent to those skilled in the art from consideration of the specification and practice disclosed herein. It is intended that the specification and examples be considered as exemplary only, with a true scope of the disclosure being indicated by the following claims and their equivalents.

What is claimed is:

1. A method for treating an inflammatory or vascular condition, said method comprising:
transdermally regulating inflammation in a target area of the body having an inflammatory or vascular condition by topically applying to the skin of the target area a therapeutically effective amount of at least one anti-VEGF containing compound.
